(19) United States
(12) Patent Application Publication
(10) Pub. No.: US 2022/0323298 A1
(43) Pub. Date: Oct. 13, 2022

(54) BATHING APPARATUS FOR THERMOTHERAPY

(71) Applicant: Tsugumitsu MATSUI, Miyazaki (JP)

(72) Inventor: Tsugumitsu MATSUI, Miyazaki (JP)

(21) Appl. No.: 17/642,747

(22) PCT Filed: Sep. 18, 2020
(86) PCT No.: PCT/JP2020/035384

§ 371 Date: Apr. 26, 2022

(30) Foreign Application Priority Data
Sep. 20, 2019 (JP) 2019-186362

(51) Int. Cl. A61H 33/02

(52) U.S. Cl. CPC A61H 33/02; A61H 2033/021; A61H 2033/022

(57) ABSTRACT

A bathing device for thermotherapy includes, attached to a bathtub, a magnetized hot water production device through which hot water is passed so as to be magnetized and an aeration device that aerates said magnetized hot water.

### TECHNICAL FIELD

The present invention relates to a bathing device for thermotherapy that warms the body with hot water having a predetermined temperature, when bathing. In particular, the present invention relates to a bathing device that efficiently generates fine bubbles using an aeration device that also generates a water flow and causes magnetic force to act on supplied water to generate fine-bubbled magnetized water, which is useful for thermotherapy.

### BACKGROUND ART

Since it is known that viruses and cancer cells are more vulnerable to heat than normal cells, thermotherapy that warms locations deep within the body to predetermined temperatures is known to be effective for preventing viruses and cancer cells from proliferating in the body. It is of note that the reasons for which viruses and cancer cells are vulnerable to heat is thought to be as follows. Specifically, human cells can survive because of homeostasis of the internal biological environment, which functions by expanding blood vessels around the cells so as to lower the body temperature, if body temperature rises to 42° C. When the body temperature rises to 42.5° C. or higher, intracellular enzymes cease to work, homeostasis of the internal biological environment is disrupted, cells are altered, and coagulation occurs, leading to death.

On the other hand, it is thought that, with cancer cells, such homeostasis of the internal biological environment does not work and, at high temperatures, for example, in excess of 41° C. to 42° C., blood vessels for cancer cells contract, so that heat tends to accumulate and cancer affected areas are subject to the effects of heat.

Conventionally, in thermotherapy, the body temperature is raised by a heating method that is thought to be able to damage only cancer cells without damaging normal cells, by maintaining the body temperature at 42° C. for a certain period of time, and the amount of heat in a heating medium such as hot water is controlled so as to maintain that body temperature for a suitable period of time. (See, for example, Patent Documents 1, 2 and 3.)

Further, when proteins in human cells begin to be denatured due to such thermotherapy, proteins known as heat shock proteins 70 (also abbreviated as HSP70) are known to be secreted, which act to perform repairs in high fever disorders and stress-related disorders in cells.

In such thermotherapy that has a desirable effect on human health, hot water and sauna devices for raising body temperature are set to a higher temperatures than normal bathing temperatures, and thus a bather may feel stress due to heat, and it is often difficult to bathe for a period of time that is sufficiently long for the body temperature to rise to the temperature required for therapy.

With such heat stress, heat perceived by the skin is felt as stress in the brain, and in order to reduce the brain stress due to bathing to as great an extent as possible, devices have been proposed, for example, in which a bathtub is covered and only the head of the patient is exposed outside the lid, or in which the entire body of the patient including the head is within the bathtub and oxygen, anesthetic gas, or the like is supplied via a tube penetrating the lid (see Patent Document 4).

It is known from this document that blue or other colored water is used for the bath, and that bathing is performed while shining colored light on the eyes, and this document also proposes that bathing is performed while listening to music, that bathing is performed while undergoing acupuncture or moxibustion, and stimulating meridians with low frequencies, lasers, or the like; and that bathing is performed while cooling the head, or mitigating brain stress through the action of two or more types of gemstones, metal ions, or magnetic fields.

However, with the bathing performed while relieving stress in accordance to the known art described above, the degree to which brain stress is relieved was not sufficient, and the brain experienced irritation from the skin, which perceives the heat, while the skin sometimes become red and inflamed, such that it could not be said that the temperature sensitivity of the bather was sufficiently suppressed.

Here, Patent Document 5 is directed to providing a bathing device for a thermotherapy device, using a bathtub, with which, even if bathing undertaken using high-temperature water exceeding 42° C., there is little irritation of the skin by heat, and the sensible temperature of the hot water is made as low as possible, allowing thermotherapy to be performed as comfortably as possible, and it is proposed that the redox potential of water with which the bathtub is filled is reduced by a water redox potential reducing device that is attached to the bathtub, such that, even with, for example, water at 43° C., the sensible temperature is lowered by at least 1 to 2° C., and heat irritation of the skin is reduced, whereby bathing is possible for relatively long periods of time at temperatures of, for example, 42 to 43° C., which are higher than water temperatures of 39 to 41° C. that are normally used for bathing.

Meanwhile, applying a magnetic field from a permanent magnet to drinking water to help in promoting health has long been practiced. For example, a mass of natural magnetite was placed in well water, left for some time, and then the water was drunk, to help in treating disease. The present inventor has focused on the fact that the earth itself is a magnetic body, and the magnetic field turns in outer space in conjunction with the autorotation thereof, and under these circumstances, living organisms have a compass-like function that reacts to geomagnetism. For example, magnetic microorganisms, such as the magnetically sensitive microorganisms discovered in 1960, have magnetite fine particles within their bodies. Furthermore, it has been found that magnetic microorganisms living in the northern hemisphere move towards the South Pole, while magnetic microorganisms living in the southern hemisphere move towards the North Pole. Thus, it can be considered that the biological functions of humans and animals are also related to internal magnetic substances.

Further, a device for producing magnetized water by applying magnetism to water is known. For example, there have been proposed: a magnetized water device comprising a holder member having an insertion hole allowing for insertion of a tube through which a fluid flows, and a plurality of magnetic field generators incorporated in the holder member (see Patent Document 6); a water magnetization device that causes magnetism to act on water flowing through a conduit, comprising plurality of magnetization chambers, in which an internal casing forming an inner magnetization flow path is formed by connecting the plurality of magnetization chambers by way of a passage, magnetizers combining a permanent magnet and a yoke plate that is magnetically attracted thereto are disposed in the magnetization chambers and, by covering the outside of the internal casing with an external casing, an external magnetization flow path is formed with which magnetized water flows out from the inner casing and is subjected to magnetizing action once again during this time (see Patent Document 7); a device in which magnetic blocks that have been magnetized by magnetic force are disposed around water pipes and the water passages of supply pipes at different positions and angles in the direction of water flow, and the passing water is swirled by the magnetic force of the magnetic blocks to produce magnetized water (see Patent Document 8); and a magnetized water production device comprising a connecting part that connects a right half body and a left half body so as to be substantially pivotally rotatable so that these can be opened and closed, in which a left-side super-strong magnet is slidably installed in the right half body (see Patent Document 9), and the like.

Mechanical aeration systems include, for example, a fine bubble generation device in which the pressure on a fluid for dissolving a gas in a liquid, in which the gas has been pressurized and dissolved in the liquid, is released by a pressure reducing valve, and is discharged as a jet from a discharge hole into a liquid tank, while fine bubbles are generated (see Patent Document 10). Furthermore, there exist fluid bed-type aeration devices, in which aeration is performed by way of fluidizing a filter medium layer with a flow of source water, using a particulate filter medium having a specific gravity that is slightly greater than the specific gravity of water, the source water passing through in the upward direction to fluidize the filter medium layer (see, for example, Patent Document 11).

There has been proposed a water purification device for fish breeding comprising: a reaction tube that is arranged so that it is submerged in water so as to be spaced apart by a predetermined amount from the bottom of a purification tank; a diffuser that is placed at the bottom of the reaction tube and is connected to an aeration pipe, which jets air into the reaction tube; a packed bed filled with a microbial metabolite including a compound having a phenol-exposing group or an elution filler made of humic substances in the reaction tube; a packed bed filled with an elution filler made of crushed stone including activated silica component such as andesite or rhyolite; and a sludge drawing tube for drawing out sludge deposited in the purification tank (see, for example, Patent Document 12).

The structure of water consists of conjugates (clusters) in which water molecules are attracted to each other by way of hydrogens, the clusters in tap water comprising, for example, an average of 12 water molecules. It has been reported that water has the property of being activated when it is affected by magnetism (magnetic force) and, therefore, when tap water is passed through a magnetic field in which magnetic force is acting, magnetized water is produced, consisting of, for example, clusters of an average of 6 water molecules. Focusing not only on the physical changes in water when a magnetic field is applied to water, but also on the chemical changes, such as the active hydrogen contained in water, active hydrogen tends to be stably present in environments in which two permanent magnet S poles face each other. Here, active hydrogen refers to hydrogen atoms in atomic form, and it has been reported that as this readily binds to active oxygen to form water, and is effective in removing active oxygen, and as a result, it can work toward improving health.

Water contains hydrogen ions (H⁺) and hydroxide ions (OH⁻), and hydrogen ions readily receive electrons from hydroxide ions and to produce atomic hydrogen, but the active hydrogen produced is very unstable because it turns into water or hydrogen ions when it comes into contact with dissolved oxygen or cations such as calcium, sodium and magnesium, in water. However, when atomic hydrogen is produced, if it is in an environment where the same poles face each other, which is to say two S poles or two N poles face each other, the atomic hydrogen can be said to be a magnetic atom with a magnetic moment direction that is not fixed, and thus, in an environment where two S poles face each other in the device of the present invention, the direction of the magnetic moment of the active hydrogen is not fixed, and the active hydrogen moves indeterminately in the water, and thus the environment is stable in terms of the active hydrogen, which results in a large amount of active hydrogen being present in the magnetized water, such that the magnetized water is considered to exhibit an effect of promoting health.

Devices for producing magnetized water by applying magnetism to water are known. For example, there have been proposed: a magnetized water device comprising a holder member having an insertion hole allowing for insertion of a tube through which a fluid flows, and a plurality of magnetic field generators incorporated in the holder member (see Patent Document 13); a water magnetization device that causes magnetism to act on water flowing through a conduit, comprising plurality of magnetization chambers, in which an internal casing forming an inner magnetization channel is formed by connecting the plurality of magnetization chambers by way of a passage, magnetizers combining a permanent magnet and a yoke plate that is magnetically attracted thereto are disposed in the magnetization chambers and, by covering the outside of the internal casing with an external casing, an external magnetization flow path is formed with which magnetized water flows out from the inner casing and is subjected to magnetizing action once again during this time (see Patent Document 14); a device in which magnetic blocks that have been magnetized by magnetic force are disposed around the water pipes and the water passages of supply pipes at different positions and angles in the direction of water flow, and the passing water is swirled by the magnetic force of the magnetic blocks to produce magnetized water (see Patent Document 15); and a magnetized water production device comprising a connecting part that connects a right half body and a left half body so as to be substantially pivotally rotatable so that these can be opened and closed, in which a left-side super-strong magnet is slidably installed in the right half body (see Patent Document 16), and the like.

Furthermore, there is a water purifier that removes chlorine from tap water, in which a pair of electrodes are arranged so that the tap water is present therebetween, comprising power supply for applying a voltage to the two electrodes, and further comprising a bubble generating means for generating bubbles in the tap water exposed to an electric field between electrodes (see Patent Document 15). There is a fine bubble generator in which water is passed through a water transit hole that runs diagonally to the conduit, to give a swirling force to the flowing water, and by inserting the tip of a jetting nozzle into a water inlet of a tube, and jetting vigorously flowing water near the water inlet, which is provided with an air suction gap, air is captured in the flowing water, which creates large bubbles, which collapse in response to the shearing force of the swirling water flow, making finer bubbles (see Patent Document 16). There is a fine bubble generator in which a threading is provided on the main body of the fine bubble generator, which matches a threading on a water supply faucet, a protrusion is provided at the introduction port of a secondary treatment chamber, and a permanent magnet is put in place to increase the fineness of bubbles and the ratio thereof (see, for example, Patent Document 17), and the like.

### Prior Art Documents

### Patent Documents

Patent Document 1: JP 06-005790 U1

Patent Document 2: JP 2006-075320 A

Patent Document 3: JP 2008-142397 A

Patent Document 4: WO 03/057098 A

Patent Document 5: JP 2007-319538 A

Patent Document 6: JP 2005-169358 A

Patent Document 7: JP 2005-040694 A

Patent Document 8: JP 11-165278 A

Patent Document 9: JP 2003-024949 A

Patent Document 10: JP 2001-347145 A

Patent Document 11: JP 03-38289 A

Patent Document 12: JP 07-041246 B

Patent Document 13: JP 11-165278 A

Patent Document 14: JP 2003-024949 A

Patent Document 15: JP 2001-232365 A

Patent Document 16: JP 2007-117799 A

Patent Document 17: JP 3145914 U

Patent Document 18: WO 03/002044 A

### SUMMARY OF THE INVENTION

### Problems to Be Solved by the Invention

In view of the aforementioned problems in the prior art, an object of the present invention is to provide a bathing device for a thermotherapy device, using a bathtub, with which, even if bathing is performed using high-temperature water exceeding 42° C., there is little irritation of the skin by heat, allowing bathing to be performed for a long period of time, and allowing thermotherapy to be performed as comfortably as possible.

### Means for Solving the Problems

In order to solve the above problems, a first feature of the bathing device for thermotherapy according to the present invention is that a magnetized hot water generator through which hot water is passed so as to be magnetized, and an aeration device that aerates said magnetized hot water are attached to a bathtub. Furthermore, a second feature is that of comprising magnetized water generation device, a water flow pump installed in stored water in a bathtub, an air aspiration tube connected to a water flow tube of the water flow pump, which aspirates atmospheric air, and an aerated water jetting nozzle, which is mounted on the end of this water flow tube, wherein: a water aspiration part is provided, which suctions hot water from the outside to the inside of the aerated water jetting nozzle; a cavitation effect due to a swirling flow generated inside the aerated water jetting nozzle and the depressurizing action produced by the swirling flow that is generated, and the venturi effect of the depressurization means releasing the pressure of the water flow in which atmospheric air has been dissolved under pressure, cause fine bubbles to be generated, and magnetized water flow containing fine bubbles is discharged while applying a magnetic field. Furthermore, a third feature is that the water aspiration part comprises a hole, a groove, or a sand-finish rough surface carved on the outer peripheral surface of the aeration tube. Further, a fourth feature is that at least two orthogonal static mixers are arranged inside the aerated water jetting nozzle. Still furthermore, a fifth feature is that it is of comprising a self-priming air aspiration device that introduces air in the form of fine bubbles by utilizing negative pressure generated with the static mixer. In addition, a sixth feature is that the aerated water jetting nozzle comprises an amorphous magnetic material.

The thermal heat treatment temperature cannot be unconditionally specified in relation to the treatment time, but a temperature of 42° C. to 44° C. is desirable, and 43° C. is optimal. If the temperature is lower than 42° C., the proliferation inhibiting effect is insufficient, and if the temperature exceeds 44° C., strong suppression of proliferating smooth muscle cells is observed, which leads to cell death even in healthy contractile smooth muscle cells having no proliferative ability, in addition to which, the proliferation of vascular endothelial cells also begins to be suppressed. Further, the thermal heat treatment time is preferably in the range of 90 to 180 minutes, and is optimally 120 minutes. If the treatment time is less than 90 minutes, the desired proliferation inhibiting effect is not observed, and even if the treatment time exceeds 180 minutes, only approximately the same degree of proliferation inhibition as at 120 minutes is observed. In addition, it has been reported that it is most desirable to add a thermal heat treatment at 43° C. for 2 hours, after allowing 2 hours to elapse following proliferative stimulation of vascular smooth muscle cells, since there is a risk of adversely affecting the proliferation of healthy contractile vascular smooth muscle cells and vascular endothelial cells that do not have the ability to proliferate (see Patent Document 18).

The present invention has the following excellent effects.

(1) By maintaining the internal body temperature at 42° C. for a certain period of time, it is possible to damage only cancer cells without injuring healthy cells.

(2) The sensible temperature is lowered by at least 1 to 2° C. with respect to the water temperature, and heat irritation of the skin is reduced, whereby bathing is possible for relatively long periods of time at high temperatures of, for example, 42 to 43° C., which are higher than water temperatures of 39 to 41° C. used for normal bathing.

(3) By forming grooves with a substantially U-shaped cross section in the side view, or a sand-finish rough surface, at the end of the Venturi tube on the aerated water discharge side, fine bubbles are further subdivided by interaction with the vortex and the amount of fine bubbles generated can be significantly increased.

(4) It is not necessary to separately provide a liquid flow generator for circulation and pumping of hot water in the bathtub, and thus it is possible to reduce the size, weight, and space.

(5) Since oxygen can be supplied and water flow can be generated at the same time with one device, and an alternating magnetic field is naturally applied by way of the air aspiration or water flow, no special power is required to generate the magnetic field.

(6) By equipping the aerated water jetting nozzle with an amorphous magnetic material, the hot water magnetization effect is more reliably achieved.

### BRIEF DESCRIPTION OF DRAWINGS

FIG. 1 is a schematic diagram showing an embodiment of a bathing device for thermotherapy according to the present invention.

FIG. 2 is a schematic diagram showing one example of an air aspiration part.

FIGS. 3(a) and 3(b) are schematic diagrams showing another example of the air aspiration part.

FIGS. 4(a) and 4(b) show an example of an ejector tube, in which FIG. 4(a) is a side sectional view, and FIG. 4(b) is a front view.

FIG. 5 is a perspective view showing an example of grooves carved into an ejector tube.

FIGS. 6(a) and 6(b) are schematic diagrams showing another embodiment of the aeration device according to the present invention.

FIG. 7 is a perspective view showing an aerated water jetting nozzle.

FIGS. 8(a) and 8(b) show an aerated water jetting nozzle in which FIG. 8(a) is a lengthwise-sectional view and FIG. 8(b) is a cross-sectional view.

FIG. 9 is a perspective view showing an embodiment of a bathing device for thermotherapy.

### BEST MODES FOR CARRYING OUT THE INVENTION

Hereafter, modes of embodiment of the present invention are described on the basis of the examples illustrated below.

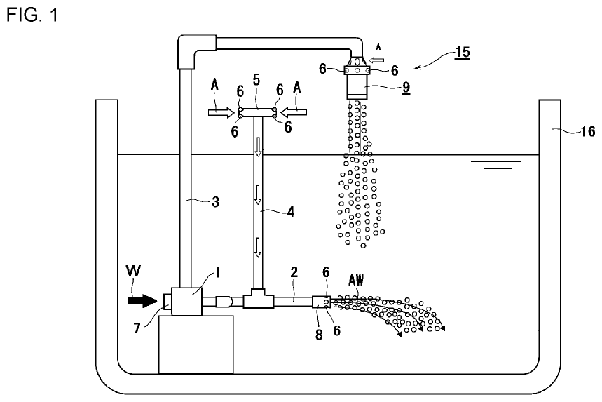

The bathing device for thermotherapy of the embodiment shown in FIG. 1 has a basic configuration equipped with a hot water supply apparatus provided with a heat-maintaining heater capable of maintaining heat in the bathtub by way of supply from a hot water boiler, in which the water temperature is adjustable, or by way of a gas or electric heater; furthermore, a magnetized aerated water generation device 15 comprises: a water flow pump 1 disposed in water, an air aspiration part 5 incorporating magnets 6, which is provided above the water, a water aspiration tube 7 and a water flow tube (Venturi tube) 3 that functions as a pressure reduction part, which are connected to the water flow pump 1, an air aspiration tube 4 connected to the water flow tube 3, and an aerated water jetting tube 8 provided with an ejector tube 2.

A hot water supply device (not shown) not only mixes cold water and hot water from a boiler in a water storage tank to adjust the hot water temperature, but usually has a recirculation path in order to recirculate a certain amount of hot water in the bathtub allowing the heat to be maintained. The recirculation path is used not only for maintaining heat but also for repeatedly passing hot water in the bathtub through the magnetized water generation device to ensure the action thereof.

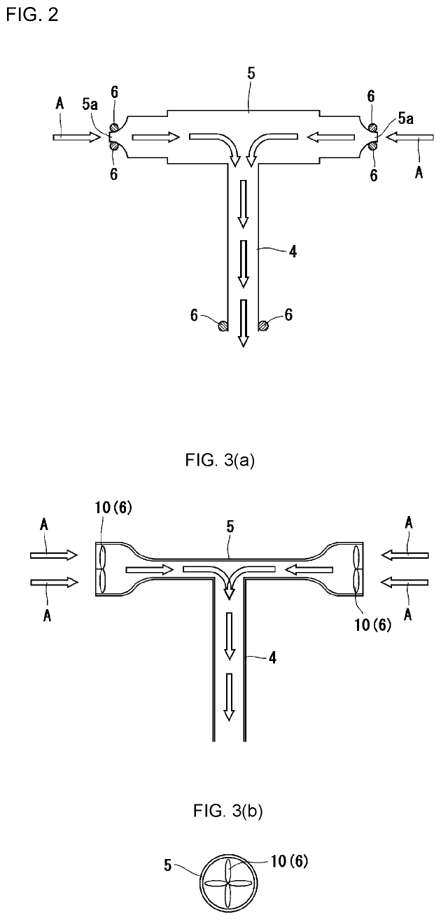

As shown in FIG. 2, both ends of the air aspiration part 5 are flattened so as to have flattened intake ports 5a or, as shown in FIGS. 3(a) and 3(b), the air aspiration parts 5 comprise propellers 10 having with integrated magnets 6 and feed aspirated atmospheric air A to the air aspiration tube 4. The water flow tube 3 utilizes the Venturi effect to depressurize and pressurize atmospheric air A aspirated from the air aspiration part 5, a long tapered portion 3a being formed which expands in diameter from the upstream side to the downstream side, and the end part (outlet portion) on the discharge side of this tapered portion 3a being formed in an outwardly oriented trumpet shape. Furthermore, the outside air A is captured to generate a gas-liquid mixed water AW in which bubbles have been mixed.

In the water flow tube 3, when the bubbles that have been generated in the flowing water pass through the tapered tube, the pressure is reduced in conjunction with the increases in the flow velocity, and these expand, whereafter, as the cross-sectional area increases, pressure is applied and these contract, and the bubbles are subdivided by the shear force generated at this time so that fine bubbles are produced.

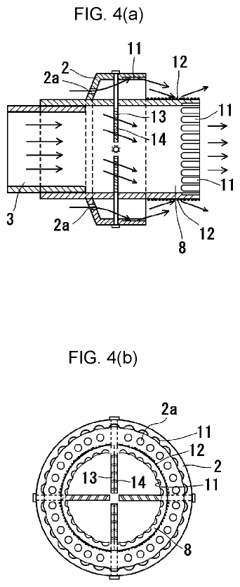

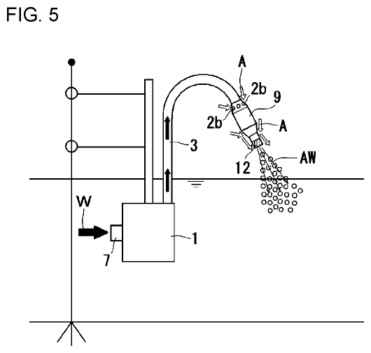

As shown in FIGS. 4(a), 4(b) and 5, the water flow is further introduced into an aerated water jetting tube 8 provided with the ejector tube 2, resulting in a swirling flow along the inner wall surface thereof, such that suction force is generated in the central portion thereof as a result of the centrifugal force generated and stored water is aspirated by way of water aspiration ports 2a in the ejector tube 2. At the same time, the swirling water flow suctions the atmospheric air, and the water flow descends toward the outlet by way of water flow guidance grooves 14 in four static mixers 13 projecting into the interior of the tube, and due to competition with the reverse suction force from below, shearing occurs in the vicinity of the outlet and fine bubbles are formed. Further, cavitation occurs, due to the turbulent flow that is produced by the swirling flow colliding with grooves 11 formed on the inner and outer peripheral surface around the outlet of the aerated water jetting tube 8, the sand-finish rough surface 12, and the like, which promotes further reduction in size of the bubbles.

That is to say, this comprises the water flow pump 1 installed in the water tank or the stored water W in a water area, the air aspiration tube 4 connected to the water flow tube 3 of the water flow pump 1, which aspirates atmospheric air, and the aerated water jetting nozzle 8, which is mounted on the end of this water flow tube 3; a water aspiration part 2a is provided, which suctions stored water from the outside to the inside of the aerated water jetting nozzle 8; a cavitation effect due a swirling flow inside the aerated water jetting nozzle 8 and the depressurizing action produced by the swirling flow that is generated, and the Venturi effect of the depressurization means releasing the pressure of the water flow in which atmospheric air has been dissolved under pressure, cause fine bubbles to be generated, and magnetized water flow containing fine bubbles is discharged while applying a magnetic field.

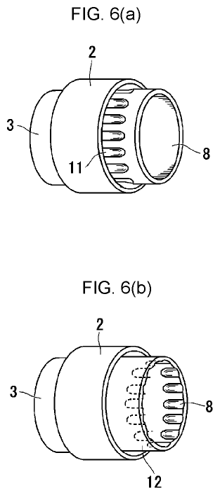

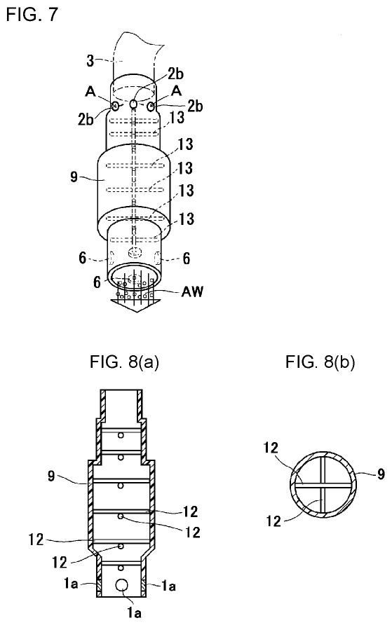

Further, the arrangement may be such that an aerated water jetting nozzle 9, which is mounted outside the stored water, and which, as shown in FIGS. 6(a), 6(b), 7, 8(a) and 8(b), is provided with a plurality of permanent magnets 6 for creating an alternating N and S magnetic field in the supply water and static mixers 13, and atmospheric air is aspirated from air aspiration holes 2b, whereby fine bubbles are produced by the cavitation action thereof. In this case, aeration can be performed at the same time as the magnetization of water, to supplement the dissolved oxygen, and this is useful, for example, as a nozzle for supplying water to a culture pond. Note, that, as shown in FIG. 10, a propeller 10 may be used instead of the static mixer 13 for aeration, and the desired effect can thereby be obtained.

Note that, a system can also be employed for the atmospheric air aspiration part, in which air is suctioned toward negative pressure generated by a static impeller, and in addition to the embodiment described above, systems in which air is suctioned from holes in a cylindrical tube along the impeller, systems in which gas jetting holes are provided on the back face of a hollow box-shaped impeller that is fixed so as to allow for passage of air with an air passage opening in a tube, systems in which gas jetting openings are provided in a spiral on a cylindrical tube along the back face of the impeller, in the rotation direction, spirally attached to the tube, and the like can be employed.

Such a self-priming air aspiration parts can generate a large amount of atmospheric air A in the form of ultrafine bubbles in flowing water. That is to say, a strong vacuum pressure can be generated in the flowing water, and atmospheric air can be suctioned into the flowing water with little energy. Furthermore, since the air is made extremely fine, oxygen dissolution efficiency can be enhanced.

Permanent magnets of 3000 gauss or more at the surface such as, for example, neodymium magnets or samarium magnets, are preferred as the permanent magnets used in the present invention. In particular, the hot water magnetizing effect is more reliably achieved with an amorphous magnetic material.

With hot water magnetized in this way, it is thought that the state of water is changed at the molecular level, such as in terms of having smaller clusters of water molecules (monomolecular water or nearly monomolecular water), and it includes inorganic ions (for example, divalent or trivalent inorganic ions, such as iron ions) eluted from mineral inorganic substances over which it has passed and with which it has made contact, thus having physical properties such that it penetrates well into various substances, as well as having properties such that the redox potential (ORP) is lower than water such as ordinary tap water.

Furthermore, it is thought that when this comes into contact with a magnetic body, ions such as Fe²⁺ and Fe³⁺are eluted, reducing the size of clusters by breaking up clusters of water molecules, or aggregating small molecules centered on the dissolved ions, and as a result of some sort of physicochemical action such as hydrogen bonds being broken and the amount of active hydrogen increasing in conjunction therewith, it is considered that the redox potential (ORP) of the hot water that has come into contact therewith is also lowered as a result.

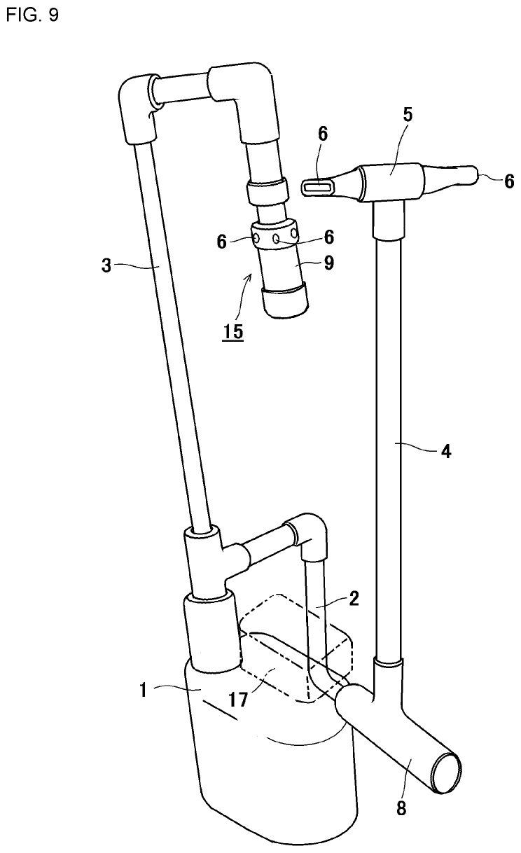

As shown in FIG. 9, the hot water supply device of the embodiment is connected to a water supply source, such as a tap water supply not shown in the figure, and the water may be directly supplied as hot water heated to a predetermined temperature (usually about 60° C.) by way of a boiler, or the temperature may be adjusted to a predetermined temperature (about 46° C.) by mixing it in a hot water storage tank with unheated water at approximately the ambient temperature, or a temperature lower than the ambient temperature, from the water supply source and, using this hot water, magnetized hot water is supplied to the bathtub 16 via a tube, by way of a pump, and via a hot water supply conduit that is also used for hot water recirculation. Note that, in the figure, 17 is a sinker weight for stable placement of the water flow pump 1 in the water.

For thermotherapy using the bathing device for thermotherapy having the configuration described above, when bathing is performed with hot water adjusted to 42.5 to 43° C., the cells are acidified, the acidification increases heat sensitivity and, due to the production of heat shock proteins (Hsp), cancer cells present non-self antigens, facilitating effective attack by immune cells which have been activated by the elevated body temperature. It is considered that the survival rate of cancer cells decreases significantly at 42.5° C. or higher, and the higher the heating temperature and the longer the time, the lower the survival rate.

Here, since the redox potential of the hot water is reduced by the aerated magnetized water generation device 15, with high-temperature water at 43° C., the sensible temperature is actually lowered by at least 1 to 2° C., allowing for thermotherapy in which bathing is performed for a long time without heat stress. In fact, with tap water supply from the water source, the redox potential of water was 500 to 550 mV (on the acidic side), while it was confirmed that this was 190 mV in the water in the bathtub in the mode of embodiment in which this was passed through the redox potential (ORP) lowering device, which is an ORP value (on the alkaline side) that is very close to the potential of −50 to 100 mV on the surface of the human body.

Furthermore, a questionnaire survey of multiple users made it clear that heat irritation of the skin is also reduced, and therefore, even with hot water at, for example, approximately 42 to 43° C., serving to maintain the internal body temperature at 39-40° C. (controlled within the range of ±0.5° C.), which is usually considered to make bathing difficult, due to heat stress, this was not perceived as very hot, and it was possible to perform bathing for 30 to 40 minutes or longer, which is extremely advantageous for treatment with the thermotherapy described above.

### INDUSTRIAL APPLICABILITY

Cells that cannot be repaired due extensive DNA damage, or that have mutations due to abnormal genes remaining due to repair mistakes are caused to commit suicide (apoptosis), which is brought about by tumor suppressor genes. The risk of carcinogenesis increases when cells that cannot undergo apoptosis remain with genetic abnormalities and mutations accumulate. With the device of the present invention, it is considered that the cancer cells that have accumulated in this way can arbitrarily be brought to an abnormal body temperature state and caused to undergo apoptosis, thereby contributing to remission.

### EXPLANATION OF THE REFERENCE NUMERALS

1 water flow pump

2 ejector tube

2a water aspiration hole

2b air aspiration hole

3 water flow tube

4 air aspiration tube

5 air aspiration part

5a air aspiration opening

6 permanent magnet

7 water aspiration tube

8 aerated water jetting tube

9 aerated water jetting nozzle

10 propeller with integrated magnet

11 groove

12 sand-finish rough surface

13 static mixer

14 flowing water guide groove

15 aerated magnetized water generation device

16 bathtub

17 sinker weight

W water flow

A atmospheric air

1. A bathing device for thermotherapy, comprising a magnetized hot water generation device configured to pass, hot water therethrough in proximity to at least one permanent magnet so as to be magnetized and an aeration device configured to aerate the magnetized hot water, the bathing device for thermotherapy being configured to be attached to a bathtub.

2. The bathing device for thermotherapy according to claim 1, wherein the aeration device comprises a water flow pump configured to be installed in hot water contained in a bathtub, an air aspiration device comprising an air aspiration tube configured to aspirated atmospheric air and communicating with a water flow tube of the water flow pump, and an aerated water jetting nozzle mounted on an end of the water flow tube, wherein: a water aspiration part is provided which communicates with the water flow tube and is configured to suction hot water from the bathtub to inside the aerated water jetting nozzle so that a cavitation effect and a depressurizing action due to a swirling flow generated inside the aerated water jetting nozzle are and a Venturi effect of the depressurization releases pressure of the water flow in which atmospheric air has been dissolved under pressure, causing fine bubbles to be generated, whereby magnetized water flow containing fine bubbles is discharged.

3. The bathing device for thermotherapy according to claim 2, wherein the water aspiration part comprises a hole, groove, or a sand-finish rough surface formed on the outer peripheral surface of the aeration tube.

4. The bathing device for thermotherapy according to claim 2, further comprising at least two orthogonal static mixers arranged inside the aerated water jetting nozzle.

5. The bathing device for thermotherapy according to claim 4, wherein the air aspiration device is self-priming and is configured to introduce air in the form of fine bubbles by utilizing negative pressure generated with the at least two orthogonal static mixers.

6. The bathing device for thermotherapy according to claim 2, wherein the aerated water jetting nozzle comprises an amorphous metal.
